(19) United States
(12) Patent Application Publication
(10) Pub. No.: US 2026/0013961 A1
(43) Pub. Date: Jan. 15, 2026

(54) DYNAMIC ADJUSTMENT OF SYSTEM FEATURES AND CONTROL OF SURGICAL ROBOTIC SYSTEMS

(71) Applicant: Covidien LP, Mansfield, MA (US)

(72) Inventor: William J. Peine, Ashland, MA (US)

(21) Appl. No.: 18/993,706

(22) PCT Filed: Jul. 11, 2023
(86) PCT No.: PCT/IB2023/057081

§ 371 Date: Jan. 13, 2025

Related U.S. Application Data
(60) Provisional application No. 63/390,660, filed on Jul. 20, 2022.

(51) Int. Cl. A61B 34/37; A61B 5/0205; A61B 34/30

(52) U.S. Cl. CPC A61B 34/37; A61B 5/0205; A61B 2034/305

(57) ABSTRACT

A surgical robotic system includes a robotic arm, a user console, and a computer. The robotic arm includes a surgical instrument, and the user console includes a handle communicatively coupled to the robotic arm or the surgical instrument. The computer is configured to receive physiological signals from a sensor monitoring a clinician, determine a physiological response of the clinician based on the received physiological signals, determine a phase or a task of a surgical procedure based on at least one of surgical sensor data or a user command to perform the task, and adjust at least one function of the surgical robotic system based on at least one of the physiological response of the clinician or the phase or task of the surgical procedure.

### BACKGROUND

Surgical robotic systems are currently being used in minimally invasive medical procedures. Some surgical robotic systems include a user console controlling a surgical robotic arm and a surgical instrument having an end effector (e.g., forceps or grasping instrument) coupled to and actuated by the robotic arm.

Surgical robotics are designed to work alongside and collaborate directly with the entire surgical team in the operating room, especially the surgeon at the console, the scrub nurse and attending surgeon at the bedside, and the circulating nurse outside of the sterile field. Due to the complexity of the system and surgical procedures, there may be a variety of emotional responses by the team while using the system. This is especially true during the learning phase, when unexpected events occur, and when the robotic arm comes close to the bedside staff standing in the sterile field.

### SUMMARY

According to one embodiment of the present disclosure, a surgical robotic system is disclosed. The surgical robotic system includes a robotic arm, a user console, and a computer. The robotic arm includes a surgical instrument, and the user console includes a handle communicatively coupled to the robotic arm or the surgical instrument. The computer is configured to receive physiological signals from a sensor monitoring a clinician, determine a physiological response of the clinician based on the received physiological signals, determine a phase or a task of a surgical procedure based on at least one of surgical sensor data or a user command to perform the task, and adjust at least one function of the surgical robotic system based on at least one of the physiological response of the clinician or the phase or task of the surgical procedure.

In an aspect, the computer may be configured to adjust at least one function of the surgical robotic system based on both of the physiological response of the clinician and the phase or task of the surgical procedure.

In an aspect, the sensor monitoring the clinician may include at least one of a wearable sensor configured to be worn by the clinician, an audio sensor configured to monitor vocal variations of the clinician, or image sensors configured to monitor images of the clinician.

In an aspect, the physiological signals may include at least one of heart rate, temperature, blood flow, or vocal variations.

In an aspect, the computer may be configured to determine whether a level of the physiological response of the clinician exceeds a preconfigured threshold and notify a second clinician in response to the level of the physiological response of the clinician exceeding the preconfigured threshold.

In an aspect, the computer may be configured to adjust at least one function of the surgical robotic system by selecting a color to illuminate an indicator light based on at least one of the physiological response of the clinician or the phase or task of the surgical procedure.

In an aspect, the computer may be configured to adjust at least one function of the surgical robotic system by changing a volume level of at least one of audible alarms or music based on at least one of the physiological response of the clinician or the phase or task of the surgical procedure.

In an aspect, the computer may be configured to adjust at least one function of the surgical robotic system by changing a maximum speed limit of the robotic arm or range of motion of the robotic arm based on at least one of the physiological response of the clinician or the phase or task of the surgical procedure.

In an aspect, the computer may be configured to adjust at least one function of the surgical robotic system by restricting movement of the robotic arm to a preconfigured workspace based on at least one of the physiological response of the clinician or the phase or task of the surgical procedure.

According to another embodiment of the present disclosure, a method for dynamic adjustment of a surgical robotic system is disclosed. The method includes determining a physiological response of the clinician based on physiological signals of a clinician sensed by a sensor, adjusting at least one function of the surgical robotic system based on the physiological response of the clinician, determining whether a level of the physiological response of the clinician exceeds a preconfigured threshold, and notifying a second clinician in response to the level of the physiological response of the clinician exceeding the preconfigured threshold.

In an aspect, the method further includes adjusting at least one function of the surgical robotic system based on the physiological response of the clinician and a phase or task of a surgical procedure.

In an aspect, the method further includes monitoring at least one of heart rate, temperature, blood flow, or vocal variations of the clinician to determine the physiological response of the clinician.

In an aspect, adjusting at least one function of the surgical robotic system based on the physiological response of the clinician may include selecting a color to illuminate an indicator light based on the physiological response of the clinician.

In an aspect, adjusting at least one function of the surgical robotic system based on the physiological response of the clinician may include changing a volume level of at least one of audible alarms or music based on the physiological response of the clinician.

In an aspect, adjusting at least one function of the surgical robotic system based on the physiological response of the clinician may include changing a maximum speed limit of a robotic arm or range of motion of the robotic arm based on the physiological response of the clinician.

In an aspect, adjusting at least one function of the surgical robotic system based on the physiological response of the clinician may include restricting movement of a robotic arm to a preconfigured workspace based on the physiological response of the clinician.

According to another aspect of the present disclosure, a non-transitory computer readable storage medium is disclosed. The non-transitory computer readable storage medium stores instructions which, when executed by a processor, causes the processor to determine a physiological response of a clinician based on physiological signals of the clinician sensed by a sensor and adjust at least one function of the surgical robotic system based on the physiological response of the clinician. The processor adjusts at least one function of a surgical robotic system based on the physiological response of the clinician by selecting a color to illuminate an indicator light based on the physiological response of the clinician, changing a volume level of at least one of audible alarms or music based on the physiological response of the clinician, changing a maximum speed limit of a robotic arm or range of motion of the robotic arm based on the physiological response of the clinician, or restricting movement of the robotic arm to a preconfigured workspace based on the physiological response of the clinician.

In an aspect, the instructions, when executed by the processor, may cause the processor to adjust at least one function of the surgical robotic system based on the physiological response of the clinician and a phase or task of a surgical procedure.

In an aspect, the instructions, when executed by the processor, may cause the processor to determine if a level of the physiological response of the clinician exceeds a preconfigured threshold, and notify a second clinician in response to the level of the physiological response of the clinician exceeding the preconfigured threshold.

In an aspect, the physiological signals include at least one of heart rate, temperature, blood flow, or vocal variations.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4A is a schematic diagram of a computer architecture of the surgical robotic system of FIG. 1 according to an embodiment of the present disclosure;

FIG. 4B is a schematic diagram of a computer architecture of a phase detector of the surgical robotic system of FIG. 1 according to an embodiment of the present disclosure; and

FIG. 5 is a flowchart illustration a method for dynamically adjusting a surgical robotic system.

### DETAILED DESCRIPTION

Embodiments of the presently disclosed surgical robotic system are described in detail with reference to the drawings, in which like reference numerals designate identical or corresponding elements in each of the several views. As used herein the term “proximal” refers to the portion of the surgical robotic system and/or the surgical instrument coupled thereto that is closer to a base of a robot, while the term “distal” refers to the portion that is farther from the base of the robot.

As will be described in detail below, the present disclosure is directed to a surgical robotic system, which includes a user console, a control tower, and one or more mobile carts having a surgical robotic arm coupled to a setup arm. The user console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

This disclosure describes a surgical robotic system that changes behavior based on the current physiological response (e.g., physiological state, emotional state, etc.) and cognitive workload of the operating room team members, such as the nurses, clinicians, and users of the system. As described in greater detail below, the physiological response can be measured using wearable sensors, audio sensors, and/or vision-based algorithms. For example, the physiological response of a person (e.g. anxiety, fear, heightened awareness, excitement, etc.) can be determined by measuring physiological signals (e.g., heart rate, temperature, blood flow in the face, vocal variations, etc.) using wearable sensors and/or sensors within the operating room which may utilize audio-based algorithms and/or vision-based algorithms. The physiological data may be utilized by the system to evaluate human-robot interactions to determine how people “feel” about working alongside a robot. For example, people may be generally apprehensive of a robot at first and then become more comfortable over time, but anxiety can spike again if the robot gets too close or does something unexpected.

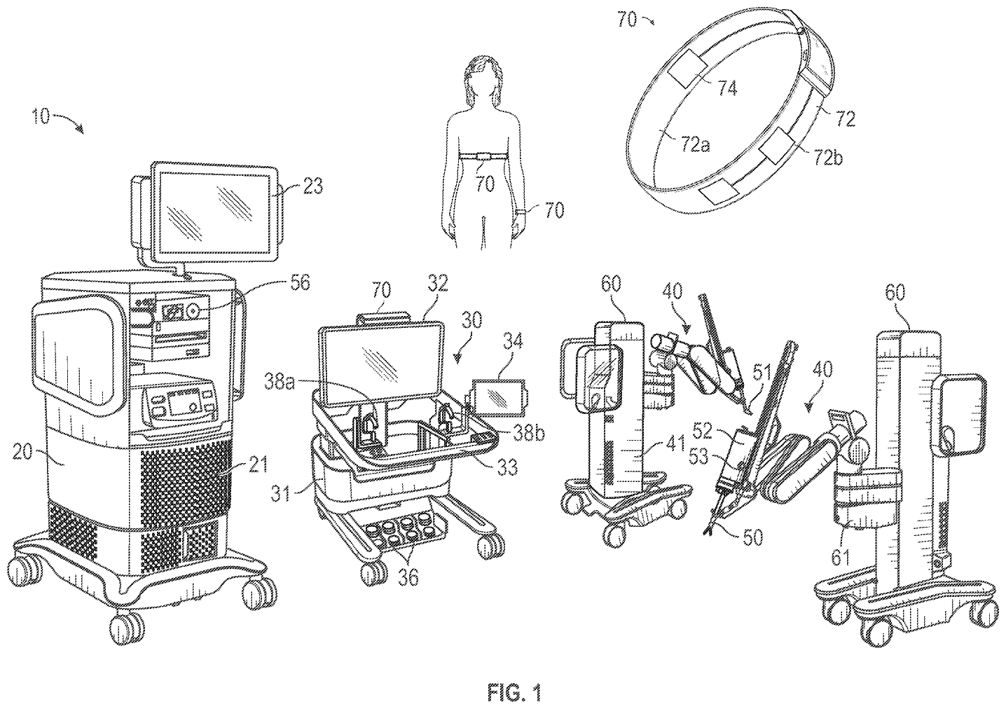

With reference to FIG. 1, a surgical robotic system 10 includes a control tower 20, which is connected to all of the components of the surgical robotic system 10 including a user console 30, one or more mobile carts 60, and one or more sensors 70 which are configured to measure physiological signals of the clinicians operating the components of the surgical robotic system 10. Each of the mobile carts 60 includes a robotic arm 40 having a surgical instrument 50 removably coupled thereto. The robotic arms 40 also couple to the mobile cart 60. The robotic system 10 may include any number of mobile carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The user console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The user console 30 also includes a plurality of user interface devices, such as foot pedals 36 and a pair of handle controllers 38a and 38b which are used by a user to remotely control robotic arms 40. The user console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the user console 30 and one or more robotic arms 40. In particular, the control tower 20 is configured to control the robotic arms 40, such as to move the robotic arms 40 and the corresponding surgical instrument 50, based on a set of programmable instructions and/or input commands from the user console 30, in such a way that robotic arms 40 and the surgical instrument 50 execute a desired movement sequence in response to input from the foot pedals 36 and the handle controllers 38a and 38b.

Each of the control tower 20, the user console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

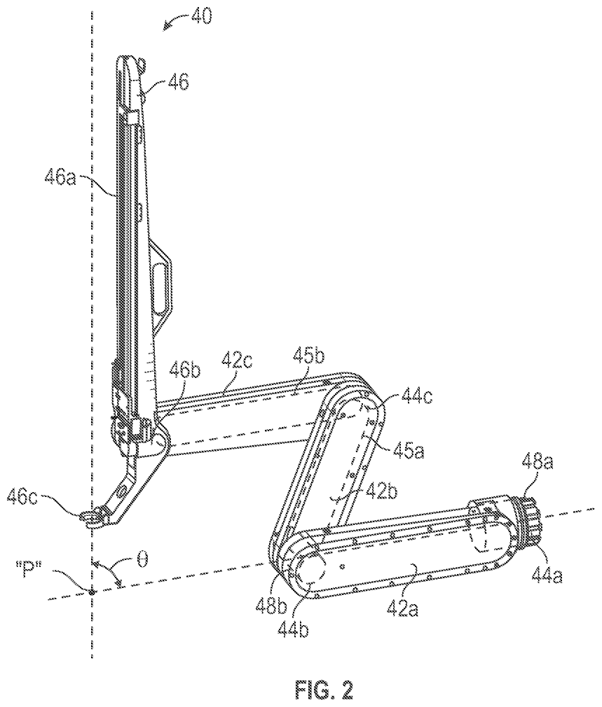

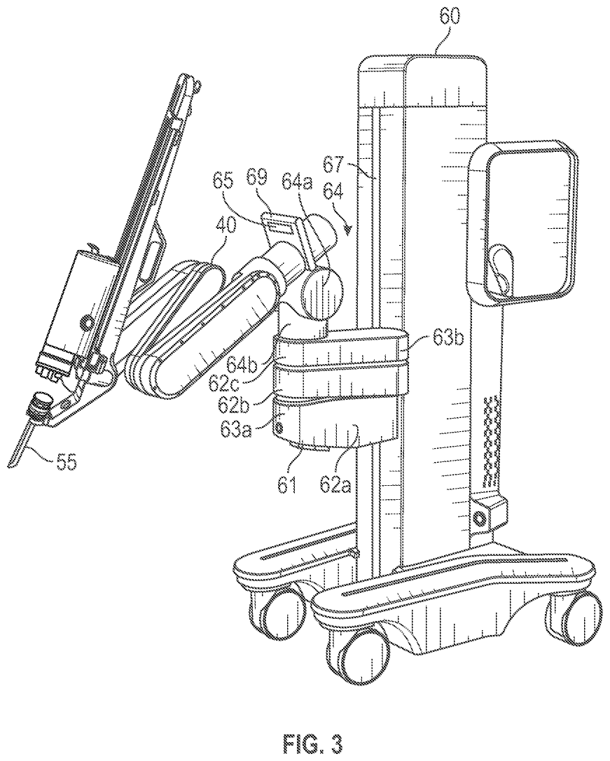

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

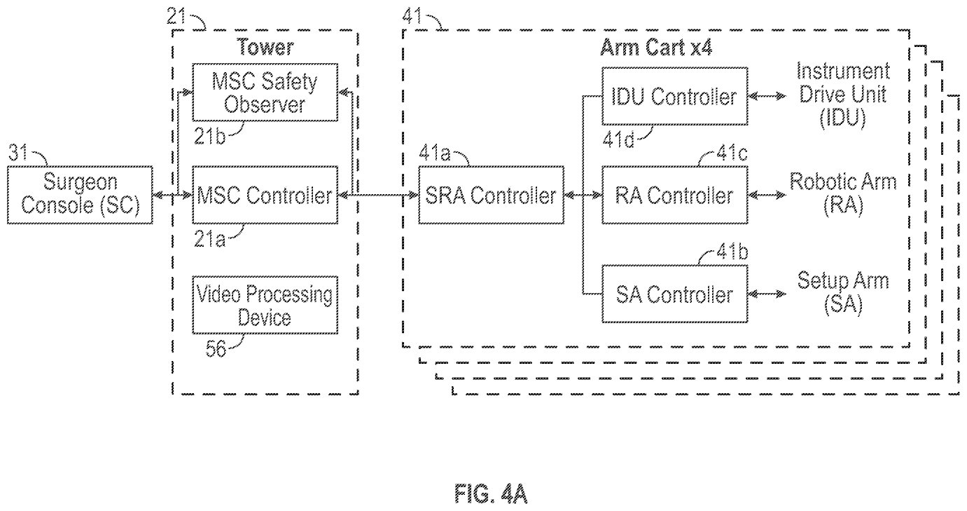

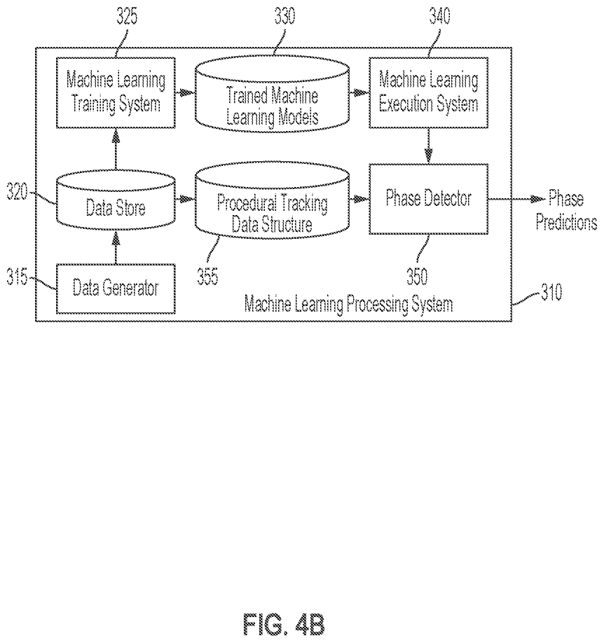

With reference to FIG. 4A, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the user console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the user console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or may be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the user console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The present disclosure provides a control algorithm, which may be embodied as software instructions executed by a controller, e.g., the controller 21a or any other suitable controller of the system 10. The control algorithm detects the current physiological response of the clinicians in real-time and automatically adjusts the control and/or functions of one or more components of the system 10 based on the detected physiological response. In aspects, the system 10 also detects the current temporal phase, step, or commanded task of the surgical procedure and automatically adjusts the control and/or functions of one or more components of the system 10 based on both of the detected physiological response and the detected phase, step, or commanded task.

The control algorithm determines the physiological response of each clinician based on physiological signals of each clinician sensed by at least one sensor 70. For example, each clinician may have a dedicated sensor 70, such as a wearable sensor 70 that measures their physiological signals (e.g., pulse, heart rate, blook-oxygen level, blood pressure, etc.) and utilizes the physiological signals or rate of change of the physiological signals to determine a physiological response of the clinician (e.g. anxiety, fear, heightened awareness, excitement, etc.). In addition to wearable sensors 70, or in lieu of wearable sensors 70, audio-based or image-based sensors 70 may be utilized to detect physiological signals of the clinicians, for example, based on vocal changes, body temperature changes, skin color changes, pupil dilation, etc.

In an aspect, the sensor 70 may be worn at one or more locations around the body or limb of a person, such as a wrist, ankle, chest, etc. The sensor 70 may be attached to the person using a band 72 or an adhesive bandage (not shown), such that the sensor 70 is in physical contact with the person allowing for measurement of sounds and other physiological signals generated by the person. When the sensor 70 is worn around the wrist, the band 72 may be formed from an elastic material, such as silicone, rubber, combinations thereof, or any suitable stretchable elastomer. The band 72 may be fitted about the wrist to induce arterial stenosis, thereby generating blood flow turbulence to enhance sound generation associated with the blood flow. When the sensor 70 is worn around chest, any suitable strap may be used, such as an adjustable and/or an elastic strap. The band 72 may be formed as a single strip. In embodiments, the band 72 may be formed from one or more strips or filaments woven in any suitable pattern.

Additionally, the sensor 70 may include one or more inner acoustic sensors 74 disposed an inner surface 72a (i.e., surface directly in contact with the person) of the band 72. The inner acoustic sensor 74 is configured to measure sounds generated within the person. The inner acoustic sensor 74 may be a microphone or any other type of acoustic transducer configured to measure sound, such as a flexible membrane transducer, a micro-electromechanical systems (MEMS) microphone, an electret diaphragm microphone, or any other microphone. When the sensor 70 is worn around the wrist, the inner acoustic sensor 74 picks up sounds generated by the blood flow, which is accentuated by the compression of the band 72. When the sensor 70 is worn around chest, the inner sensor 74 picks up sounds generated by the heart, digestive system, respiratory system of the person. In embodiments, the inner sensor 74 may be a heartrate monitor such as an electrocardiography (“ECG”) sensor. The ECG sensor is configured to measure electrical activity of the heart and is disposed on the chest of the person. The inner sensor 74 may also be a photoplethysmography-based sensor which uses optical sensors to detect volume of blood flow. Since the optical sensor measures blood flow, the inner sensor 74 may be placed at any suitable location having sufficient blood flow.

The control algorithm detects the phase, step, or commanded task based on one or more sensors 70 coupled to one or more components of the system 10, one or more sensors 70 placed within the surgical setting, commands received from a user (e.g., commands received via any input devices such as handle controllers 38a and 38b or user interfaces), and/or inputs to and outputs form one or more other controllers of system 10. Based on the sensed data and/or received commands, the control algorithm determines the phase of the procedure, for example, initial surgical room preparation, robotic arm positioning, surgical instrument attachment, initial dissection, fine manipulation/dissection, grasping, suturing, etc. and may also categorize the phase or task, for example, as a safety-critical task. Depending on the procedure type, the control algorithm may also determine the next phase or task that follows the phase or task and perform a function based on the next phase or task. That is, the control algorithm of the system 10 could pre-emptively adjust information displays for the operating room team to optimize preparation of the next phase (e.g., prepare relevant instrumentation, notify relevant users that will be required in the next step, etc.).

With brief reference to FIG. 4B, machine learning models can detect surgical actions, surgical phases, anatomical structures, surgical instruments, and various other features from the data associated with a surgical procedure. The detection can be performed in real-time in some examples. Alternatively, or in addition, the algorithm analyzes the surgical data, i.e., the various types of data captured during the surgical procedure, in an offline manner (e.g., post-surgery). In one or more examples, the machine learning models detect surgical phases based on detecting some of the features such as the anatomical structure, surgical instruments, etc.

While some techniques for predicting a surgical phase in the surgical procedure are described herein, it should be understood that any other technique for phase prediction can be used without affecting the aspects of the technical solutions described herein. In some examples, the machine learning processing system 310 includes a phase detector 350 that uses the machine learning models to identify a phase within the surgical procedure. The machine learning models may be learned from a machine learning training system 325 employing a data generator 315 which can access a data store 320 to record data, including images and videos collected during one or more medical procedures for intelligent training of the machine learning processing system 310.

Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by a user such as a clinician. The procedural tracking data structure 355 identifies a set of potential phases that can correspond to a part of the specific type of procedure.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure or a clinician within the surgical setting. For example, the biological state can indicate a complication (e.g., blood clots, clogged arteries/veins, etc.), pre-condition (e.g., lesions, polyps, etc.). In some examples, the machine learning models 330 are trained to detect an “abnormal condition,” such as hemorrhaging, arrhythmias, blood vessel abnormality, etc.

Each node within the procedural tracking data structure 355 can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, phase detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The phase detector 350 outputs the phase prediction associated with a portion of the video data that is analyzed by the machine learning processing system 310. The phase prediction is associated with the portion of the video data by identifying a start time and an end time of the portion of the video that is analyzed by the machine learning execution system 340. The phase prediction that is output can include an identity of a surgical phase as detected by the phase detector 350 based on the output of the machine learning execution system 340. Further, the phase prediction, in one or more examples, can include identities of the structures (e.g., instrument, anatomy, etc.) that are identified by the machine learning execution system 340 in the portion of the video that is analyzed. The phase prediction can also include a confidence score of the prediction. Other examples can include various other types of information in the phase prediction that is output.

In an aspect, the control algorithm changes the range of motion of one or more joints 44a, 44b, 44c, of the robotic arms 40 based on one or both of the detected physiological response of one or more clinicians and the detected task. In certain situations, the mobile carts 60 may be placed closer together with a smaller range of motion to avoid collisions, with the range of motion shifting in real-time during the procedure depending on the surgical task and location of the current operative surgical site. The joint limits may be setup as hard boundaries or soft boundaries that decrease speed limits or adjust the limit of other arm joints as the user moves away from the normal working range. The control algorithm may also change the speed limit of the robotic arms 40 or components of the surgical instrument 50 based on the detected physiological response of one or more clinicians and/or the detected surgical task. In certain embodiments, the control algorithm increases the speed limit of the robotic arms 40 during initial dissection and decreases the speed limits for safety-critical tasks or small scale tasks (e.g., fine dissection, suturing, etc.) or when a heightened physiological response is detected (e.g., heightened cognitive load, stress, tasks requiring intense focus, etc.). In such an embodiment, the control algorithm may detect that the task is an initial dissection based on the elapsed time from the start of the procedure, based on the specific surgical instrument 50 being controlled, based on an explicit input by the user indicating that the action being performed is initial dissection, based on motion sensors, based on the position of the robotic arm 40 or surgical instrument 50 relative to the patient, based on one or more other sensors within the operating room, or any other such means or combinations thereof. Once the control algorithm determines that the task is an initial dissection, the control algorithm sets the speed limit of the robotic arm 40 and/or surgical instrument 50 accordingly. In one such configuration, the control algorithm dynamically reduces the speed limit of the robotic arm 40 and/or surgical instrument 50 as the surgical instrument 50 approaches the patient, that is, based on the distance of the surgical instrument 50 relative to the patient.

The control algorithm may also dynamically modify the motion scaling between the handle controllers 38a and 38b and the surgical instrument 50 based on the detected physiological response of one or more clinicians and/or the detected phase or task, for example, with smaller scaling for tasks that require large sweeping motions (e.g., moving the bowel around) and higher scaling for tasks that require small careful motions (e.g., fine dissection or suturing). In an aspect, the control algorithm may scale to accommodate for patient-specific information (e.g., accounting from BMI). The control algorithm may alternate mapping between the handle controllers 38a and 38b and the tip of the surgical instrument 50 when suturing to allow easier actuation of the motions required (amplified rotations, remapping of angles so that more comfortable hand positions are used, etc.). Additionally, or alternatively, the control algorithm may change the PD control gains in the joints 44a, 44b, 44c of the robotic arms 40 to improve tracking accuracy while reducing speed limits to avoid instability, changing the velocity compensation levels to improve the dynamic response of the system 10 or optimally compensate for backlash.

In educational or training configurations, when the system 10 is being used by a user for the first time, the control algorithm may alter the allowed range of motion of the surgical instrument 50 inside the patient based on the detected physiological response of one or more clinicians and/or the current surgical phase, step, or task. This improves safety while users are still learning the system 10 and performing initial cases, where it is likely that heightened anxiety will be present. Experts may use this feature to provide safety zones so they can operate faster with less worry about accidental injury. In such a configuration, the control algorithm may create safety zones around a patient, or a user may designate safety zones around the patient, where the control algorithm will reduce the range of motion and/or speed of the robotic arm 40 and/or surgical instrument 50, when the surgical instrument 50 approaches or enters the safety zone. In aspects, the size or shape of the safety zone, or the degree to which the range of motion or speed limit is changed, may be dynamically adjusted by the control algorithm based on the detected physiological response of one or more clinicians.

The control algorithm may also cause the system 10 to initiate certain applications, modify graphical user interfaces and items displayed, control illumination (e.g., color, brightness level, etc.) of one or more lights, and/or display pop-ups based on the detected physiological response of one or more clinicians and/or the detected phase or task. For example, a so-called follow-me mode, where camera angles are adjusted to follow movements of another surgical instrument, or other camera control schemes could automatically change depending on the phase or task, or physiological response of a clinician, to optimize the surgical field of view or apply some specific presets on distance between the camera 51 and the site. Additionally, or alternatively, the control algorithm may change notification settings (e.g., reduce the volume, silence, reroute, reconfigure, etc.) for particular users of the system 10 when the control algorithm detects a change in a clinician's physiological response. For example, the stress level or cognitive load of the clinician may cause the algorithm to reduce the volume level of alarms from the robotic system 10, silence some alarms that are not critical or require immediate action, change the graphical user interface to provide only critical information for the current task, dim the lights in the room, reduce the volume of the music, change the color or lights on one or more components of the system 10 to notify clinician's to focus in or reduce idle chatter, and/or add notifications on a graphical user interface to alert clinicians (e.g., the bedside team) to focus in and pay attention.

The control algorithm may determine when the detected physiological response of one or more clinicians is outside expected metrics or behaviors. For example, the control algorithm may detect a level of a physiological response of a clinician and compare that level to a preconfigured threshold (e.g., a baseline level) which may be fixed or which may dynamically change based on the phase of the surgical procedure. In such instances where the control algorithm detects that a physiological response of one or more clinicians is outside an expected metric or behavior, the control algorithm may notify one or more other clinicians (e.g., a supervisor) of the unexpected physiological response of the clinician. Additionally, the control algorithm may modify the function of the foot pedals 36 or buttons associated with the handle controllers 38a and 38b, or the function of other control devices, based on a determination that the physiological response of one or more clinicians falls outside of an acceptable range.

In aspects, the stress or cognitive load of the bedside staff is measured and relayed back to the surgeon for better situational awareness. This information could be displayed on the user console 30. A color indicator light on the user console 30 or in the graphical user interface could convey the information (e.g. green=happy, blue=bored, yellow=slight anxiety, red=high anxiety, etc.).

The control algorithm may enable live links or initiate remote communications to remote control systems or devices that enable feedback from mentors or other specialists based on the detected physiological response of the clinician, for example, to respond to inadvertent injury to a critical structure or organ that requires another consultant's guidance when the control algorithm determines that the clinician's physiological response would benefit from such assistance.

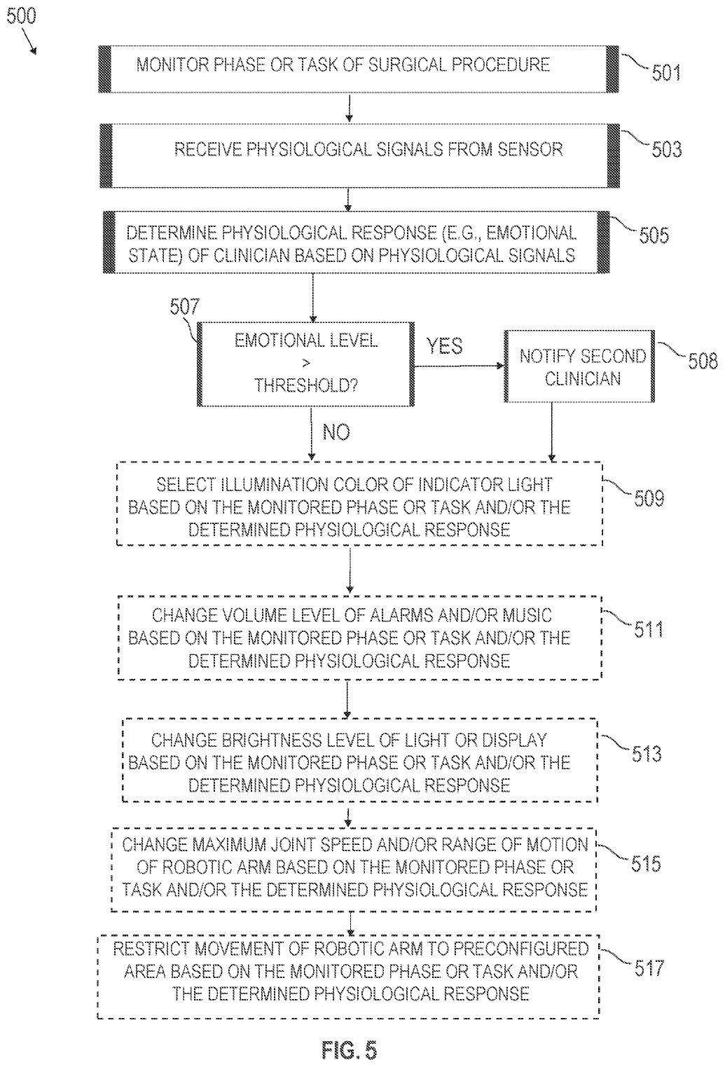

FIG. 5 illustrates a method for dynamically adjusting or controlling components of the surgical robotic system 10, and is illustrated as method 500. Method 500 may be an algorithm executed by a processor or controller of any component, or combination of components, of surgical robotic system 10. Although method 500 is illustrated and described as including specific steps, and in a specific order, method 500 may be carried out with fewer or more steps than described and/or in any order not specifically described.

Method 500 begins at step 501 where the phase, step, or task of the surgical procedure is monitored. The phase, step, or commanded task is determined based on one or more sensors coupled to one or more components of the system 10, one or more sensors placed within the surgical setting, commands received from a user (e.g., commands received via any input devices such as handle controllers 38a and 38b or user interfaces), and/or inputs to and outputs form one or more other controllers of system 10.

In step 503, physiological signals are received from at least one sensor 70 monitoring the clinicians. The physiological signals can include one or more of the clinician's heart rate, pulse, blood oxygen level, temperature, movement rate, redness, vocal behavior, or any other such signals, for example, electroencephalography (EEG), electrocardiography (ECG), electromyograms (EMG), galvanic skin response (GSR), electrodermal activity (EDA), or skin conductance (SC), heart rate (HR), heart rate variability (HRV), photoplthysmography (PPG), blood pressure (BP), respiration rate (RR), skin temperature, eye activity, pupil dilation, motion analysis, facial thermal infrared data, blood volume pulse (BVP), respiratory effort, body temperature, electrooculography (EOG), facial expressions, body posture, gesture analysis, etc. As described above, the system 10 may utilize sensors worn by the user and/or external sensors 70 within the operating room such as audio sensors 70 or video sensors 70 monitoring the physiological parameters of the clinicians.

In step 505, the control algorithm determines a physiological response, or a level associated with a physiological response of the clinician based on the physiological signals received in step 503. For example, the control algorithm may determine that the clinician is experiencing heightened anxiety, fear, excitement, stress and/or awareness. Step 505 may additionally include assigning a value associated with the determined physiological response (e.g., via a look-up table).

In step 507, the control algorithm determines whether the value of the physiological response determined in step 505 exceeds a threshold. For example, in step 507, the control algorithm may determine whether the physiological response of one or more clinicians falls outside of a preconfigured expected range (e.g., when detected phases, tasks, or events are outside expected metrics or behaviors). In aspects, the control algorithm dynamically adjusts the preconfigured expected range based on the current phase of the surgical procedure. When it is determined that the current physiological response is outside of an expected metrics range, method 500 may optionally proceed to step 508. In step 508, the control algorithm notifies a second clinician (e.g. a supervisor) of the unexpected or compromised physiological response of a clinician associated with the procedure. Following step 508, or following step 507 when no notification is warranted, method 500 proceeds to one or more of step 509, step 511, step 513, step 515, and/or step 517.

In step 509, the control algorithm selects a color to illuminate an indicator light based on at least one of the physiological response of the clinician or the phase or task of the surgical procedure. For example, the control algorithm may select to illuminate an indicator light red when heightened anxiety of one or more clinicians is detected. In another example, the control algorithm may select to illuminate an indicator light in cool colors, such as blues and green, when heightened anxiety of one or more clinicians is detected in an attempt to sooth the heightened anxiety. In step 511, the control algorithm changes a volume level of at least one of audible alarms or music based on at least one of the physiological response of the clinician or the phase or task of the surgical procedure. For example, the control algorithm may reduce the volume of notifications, silence notifications, or reconfigure audible notifications as visual notifications (e.g., pop-ups on a display, illuminating lights, etc.) when the control algorithm detects heightened anxiety of one or more clinicians.

In step 513, the control algorithm changes a brightness level of a light or a display based on at least one of the physiological response of the clinician or the phase or task of the surgical procedure. For example, the control algorithm may increase a brightness level when the control algorithm detects that one or more clinicians is fatigued. Additionally, or alternatively, for example, the control algorithm may reduce the brightness levels of one or more lights or displays when the control algorithm detects heightened anxiety of one or more clinicians.

In step 515, the control algorithm changes a maximum speed limit of the robotic arm 40 (or its components) or range of motion of the robotic arm 40 (or its components) based on at least one of the physiological response of the clinician or the phase or task of the surgical procedure. For example, the control algorithm may reduce the speed limit or range of motion of the robotic arm 40 when the control algorithm detects heightened anxiety of one or more clinicians.

In step 517, the control algorithm restricts movement of the robotic arm 40 (or its components) to a preconfigured area based on at least one of the physiological response of the clinician or the phase or task of the surgical procedure. For example, the control algorithm may disable movement of the robotic arm 40 within areas that fall outside of a preconfigured zone defined as safely away from a patient when the control algorithm detects heightened anxiety of one or more clinicians.

While the disclosure contemplates and discloses applicability to wireless communication of data, it is contemplated and within the scope of the disclosure for the principles disclosed herein to apply equally to dedicated wired communications and or hybrid wired and wireless communications.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system, comprising:
a robotic arm including a surgical instrument coupled thereto;
a user console including:
a handle communicatively coupled to at least one of the robotic arm or the surgical instrument; and
a computer configured to:
receive physiological signals from a sensor monitoring a clinician;
determine a physiological response of the clinician based on the received physiological signals;
determine a phase or a task of a surgical procedure based on at least one of surgical sensor data or a user command to perform the task; and
adjust at least one function of the surgical robotic system based on at least one of the physiological response of the clinician or the phase or task of the surgical procedure.

2. The surgical robotic system of claim 1, wherein the computer is configured to adjust at least one function of the surgical robotic system based on both of the physiological response of the clinician and the phase or task of the surgical procedure.

3. The surgical robotic system of claim 1, wherein the sensor monitoring the clinician includes at least one of a wearable sensor configured to be worn by the clinician, an audio sensor configured to monitor vocal variations of the clinician, or image sensors configured to monitor images of the clinician.

4. The surgical robotic system of claim 1, wherein the physiological signals include at least one of heart rate, temperature, blood flow, or vocal variations.

5. The surgical robotic system of claim 1, wherein the computer is configured to:
determine a level of the physiological response of the clinician;
determine whether the level of the physiological response of the clinician exceeds a preconfigured threshold; and
notify a second clinician in response to the level of the physiological response of the clinician exceeding the preconfigured threshold.

6. The surgical robotic system of claim 1, wherein the computer is configured to adjust at least one function of the surgical robotic system by selecting a color to illuminate an indicator light based on at least one of the physiological response of the clinician or the phase or task of the surgical procedure.

7. The surgical robotic system of claim 1, wherein the computer is configured to adjust at least one function of the surgical robotic system by changing a volume level of at least one of audible alarms or music based on at least one of the physiological response of the clinician or the phase or task of the surgical procedure.

8. The surgical robotic system of claim 1, wherein the computer is configured to adjust at least one function of the surgical robotic system by changing a maximum speed limit of the robotic arm or range of motion of the robotic arm based on at least one of the physiological response of the clinician or the phase or task of the surgical procedure.

9. The surgical robotic system of claim 1, wherein the computer is configured to adjust at least one function of the surgical robotic system by restricting movement of the robotic arm to a preconfigured workspace based on at least one of the physiological response of the clinician or the phase or task of the surgical procedure.

10. A method for dynamic adjustment of a surgical robotic system, comprising:
determining a physiological response of a clinician based on physiological signals of the clinician sensed by a sensor;
adjusting at least one function of the surgical robotic system based on the physiological response of the clinician;
determining whether a level of the physiological response of the clinician exceeds a preconfigured threshold; and
notifying a second clinician in response to the level of the physiological response of the clinician exceeding the preconfigured threshold.

11. The method of claim 10, further comprising adjusting at least one function of the surgical robotic system based on the physiological response of the clinician and a phase or task of a surgical procedure.

12. The method of claim 10, further comprising monitoring at least one of heart rate, temperature, blood flow, or vocal variations of the clinician to determine the physiological response of the clinician.

13. The method of claim 10, wherein adjusting at least one function of the surgical robotic system based on the physiological response of the clinician includes selecting a color to illuminate an indicator light based on the physiological response of the clinician.

14. The method of claim 10, wherein adjusting at least one function of the surgical robotic system based on the physiological response of the clinician includes changing a volume level of at least one of audible alarms or music based on the physiological response of the clinician.

15. The method of claim 10, wherein adjusting at least one function of the surgical robotic system based on the physiological response of the clinician includes changing a maximum speed limit of a robotic arm or range of motion of the robotic arm based on the physiological response of the clinician.

16. The method of claim 10, wherein adjusting at least one function of the surgical robotic system based on the physiological response of the clinician includes restricting movement of a robotic arm to a preconfigured workspace based on the physiological response of the clinician.

17. A non-transitory computer readable storage medium storing instructions which, when executed by a processor, cause the processor to:
determine a physiological response of a clinician based on physiological signals of the clinician sensed by a sensor;
adjust at least one function of a surgical robotic system based on the physiological response of the clinician by:
selecting a color to illuminate an indicator light;
changing a volume level of at least one of audible alarms or music;
changing a maximum speed limit of a robotic arm or range of motion of the robotic arm; or
restricting movement of the robotic arm to a preconfigured workspace.

18. The non-transitory computer readable storage medium of claim 17, wherein the instructions, when executed by the processor, cause the processor to adjust at least one function of the surgical robotic system based on the physiological response of the clinician and a phase or task of a surgical procedure.

19. The non-transitory computer readable storage medium of claim 17, wherein the instructions, when executed by the processor, cause the processor to:
determine if a level of the physiological response of the clinician exceeds a preconfigured threshold; and
notify a second clinician in response to the level of the physiological response of the clinician exceeding the preconfigured threshold.

20. The non-transitory computer readable storage medium of claim 17, wherein the physiological signals include at least one of heart rate, temperature, blood flow, or vocal variations.
